(19) United States
(12) Patent Application Publication
(10) Pub. No.: US 2017/0245841 A1
(43) Pub. Date: Aug. 31, 2017

(54) SYSTEMS AND METHODS FOR IMPROVED TISSUE SAMPLING

(71) Applicant: Boston Scientific Scimed, Inc., Maple Grove, MN (US)

(72) Inventors: Sean P. Fleury, Minneapolis, MN (US); Gary J. Leanna, Holden, MA (US); Michael Powers, Pepperell, MA (US); Anne Sluti, Watertown, MA (US); Joseph A. Levendusky, Groton, MA (US)

(21) Appl. No.: 15/440,736

(22) Filed: Feb. 23, 2017

Related U.S. Application Data
(60) Provisional application No. 62/299,899, filed on Feb. 25, 2016.

(51) Int. Cl. A61B 10/04; A61B 10/02

(52) U.S. Cl. CPC A61B 10/04; A61B 10/02

(57) ABSTRACT

This disclosure concerns systems and methods for tissue sampling using ultrasound guidance. Systems according to the various embodiments of the disclosure include a needle, for instance a biopsy needle, disposed coaxially about small gauge ultrasound probe. These systems are useful in a variety of procedures including pulmonary nodule sampling.

### CROSS REFERENCE TO RELATED APPLICATIONS

This application claims the benefit of priority to U.S. Provisional Application Ser. No. 62/299,899, filed Feb. 25, 2016, the disclosure of which is herein incorporated by reference in its entirety.

### FIELD OF THE DISCLOSURE

This application relates to the field of medical devices and medical procedures. More particularly, the application is related to devices and methods for ultrasound guided biopsy sampling.

### BACKGROUND

Biopsies are a group of medical diagnostic tests used to determine the structure and composition of tissues or cells. In biopsy procedures, cells or tissues are sampled from an organ or other body part to permit their analysis, for example under microscope. Generally, if an abnormality is found through superficial examination such as palpation or radiographic imaging, a biopsy can be performed to determine the nature of the suspected abnormality.

Biopsies can be performed on a number of organs, tissues, and body sites, both superficial and deep, and a variety of techniques may be utilized depending on the tissue or body part to be sampled, the location, size, shape and other characteristics of the abnormality, the number of abnormalities, and patient preference. FNA (fine needle aspiration) is typically performed to sample deep tissues such as the kidney using a fine gauge needle (22 or 25 gauge) inserted percutaneously or through an endoscope under ultrasound guidance (EUS-FNA). By contrast, surgical biopsy is generally performed as an open procedure and can be either excisional (removal of an entire lesion) or incisional (removal of a piece of a lesion).

Surgical biopsies generally permit removal of more tissue than fine needle biopsies and, thus, are less prone to misdiagnosis. Open surgical procedures are significantly more expensive than needle biopsies, require more time for recuperation, require sutures, can leave a disfiguring scar, require anesthesia, carry a small risk of mortality, and can result in bleeding, infection and wound healing problems.

Fine needle biopsies, however, carry risks of their own: the relatively small quantities of tissue sampled may not be representative of the region of interest from which it is taken, particularly when that region of interest is very small or very hard. Additional difficulties arise in the context of ultrasound-guided fine needle biopsies: fine-gauge biopsy needles are typically stiffer, and less prone to deflection, than the catheter-based endoscopic ultrasound transducers used to guide them in some EUS-FNA procedures; thus, while it may be possible to guide the transducer to a site of interest, it may not be possible to accurately sample it if the needle is too stiff to navigate the same path through the tissue. In addition, current practice involves “blind” actuation of the biopsy needle, which may result in damage to non-target tissues.

The difficulties of fine needle biopsies are magnified in the context of pulmonary nodule sampling, where breathing rhythm cause nodules, probes and needles to move relative to one another. It would be particularly desirable in this setting to be able to visualize the nodule and needle in real time during patient respiration to ensure accurate needle tracking and sampling.

### SUMMARY OF THE DISCLOSURE

The present disclosure, in its various aspects, provides improved systems and methods for fine needle aspiration in which tracking of needles and ultrasound transducers is improved and imaging of tissue acquisition in near-real time is possible.

In one aspect, the present disclosure relates to a system that includes a needle and an ultrasound probe slidably disposable in a lumen of the needle. The ultrasound probe can emit ultrasound frequencies of between 1 megahertz (MHz) and 400 MHz, for instance 20-400 MHz or over 20 MHz. In various cases, the ultrasound probe and/or the needle is disposable and/or includes a radiopaque material. The needle, optionally or additionally, is stiffer than the ultrasound probe, and/or may have a gauge between 15 and 25. The system, in some cases, also includes a device such as an endoscope, trocar, cannula or access sheath which defines a lumen sized to permit insertion of the needle. In some instances, the system includes a bronchoscope having at least one working channel sized for insertion of the needle. The needle may be actuatable to extract a biopsy from a tissue of a patient, and at least one of the needle and the ultrasound probe can be connected to an actuator for extracting such a biopsy, for instance within a space in the lumen of the needle that is distal to the ultrasound probe, when the probe is retracted away from the distal tip of the needle. In some cases, the biopsy from the tissue of the patient is positioned within the lumen of the needle between a distal end of the lumen and the ultrasound probe slidably disposed within the lumen. The needle may also be stiff enough to be inserted through a bronchial wall (or other cartilaginous structure such as the trachea, esophagus, etc.). In various instances, the needle and/or ultrasound probe includes a radiopaque material for visualization under fluoroscopy. Systems according to this aspect of the disclosure may be useful in medicine generally, and may be of particular use in the sampling of pulmonary nodules.

In another aspect, the present disclosure relates to a method of treating a patient, for example, inserting a biopsy needle and ultrasound probe through a bronchoscope into a bronchial tube of a patient, wherein the ultrasound probe is slidably disposed within a lumen of the biopsy needle; advancing the ultrasound probe and the biopsy needle to a site where a biopsy is desired; retracting the ultrasound probe into the biopsy needle, thereby creating a space into which a tissue sample can fit within the biopsy needle, and actuating the biopsy needle, thereby acquiring a tissue sample in the space. In the example of bronchial biopsy, the step of advancing the ultrasound probe and biopsy needle to a site where a biopsy is desired optionally includes advancing the biopsy needle and ultrasound probe through a wall of the bronchial tube and through a lung tissue, during which process the ultrasound probe is optionally disposed within the biopsy needle. The process of advancing the ultrasound probe and biopsy needle to the biopsy site can also include visualizing the biopsy needle under fluoroscopy and/or visualizing the site using an ultrasound signal generated by the ultrasound probe. The site being biopsied can be a pulmonary nodule, in which case the method also optionally involves contacting the pulmonary nodule or lung tissue near the pulmonary nodule with the biopsy needle, thereby altering the shape of the pulmonary nodule (for example, rendering an eccentric nodule more concentric to facilitate sampling and/or removal).

In yet another aspect, the present disclosure relates to a system that includes a bronchoscope with a working channel, a biopsy needle slidably disposable within the working channel, and an ultrasound probe slidably disposable within a lumen of the biopsy needle. As described above, the system has several optional features: the probe may emit ultrasound frequencies between 20 and 400 MHz; the probe and/or needle may be disposable and/or radiopaque, the needle may be stiffer than the probe, and the needle may have a gauge between 15 and 25.

### DRAWINGS

Aspects of the disclosure are described below with reference to the following drawings in which like numerals reference like elements, and wherein:

FIG. 1 is a photograph of a prototype biopsy system according to an embodiment of the present disclosure.

FIG. 2 is a schematic depiction of an exemplary biopsy system according to an embodiment of the present disclosure, in which an ultrasound probe is coaxially disposed within a fine biopsy needle, which in turn is slidably disposed in a sheath, catheter, or working channel of an endoscope such as a bronchoscope.

FIG. 3 is a schematic depiction of an exemplary biopsy system according to an embodiment of the present disclosure in which the needle has been retracted over the ultrasound probe.

FIG. 4 is a schematic depiction of an exemplary biopsy system according to an embodiment of the present disclosure in which the ultrasound probe is retracted and the needle advanced for deployment.

FIGS. 5A and 5B are schematic depictions of tortuous (5A) approaches for nodule sampling used today, and a more direct “tunneling” approach (5B) made possible by the systems and methods of the present disclosure.

Unless otherwise provided in the following specification, the drawings are not necessarily to scale, with emphasis being placed on illustration of the principles of the disclosure.

### DETAILED DESCRIPTION

In general, the biopsy systems of the present disclosure arrange an ultrasound probe concentrically within a biopsy needle, an arrangement which prevents kinking and breaking of the probe, and which eases insertion and navigation of the probe to a biopsy site. The concentric arrangement of the ultrasound probe within the biopsy needle also permits real-time verification that the needle is correctly placed. Finally, needles of the present disclosure generally utilize larger diameter (smaller gauge) needles than are typically used for FNA, to accommodate the ultrasound probe. This has a number of useful consequences: first, the larger needle obviates the need for a sheath to be placed over the needle and probe. Second, the larger needles are able to harvest more tissue than those currently used in the art. And third, the larger needles are stiffer than those currently used in the art, which permits both the needle and ultrasound probe to be introduced through a scope such as a bronchoscope and then tunneled through tissue to sample deep lying structures and/or to avoid tortuous anatomy. In fact, the stiffness of the larger needle can, in some cases, advantageously permit a user to move tissue, such as lung tissue near eccentric nodules (e.g. nodules in which the main cystic component is disposed near an edge or peripheral portion of the nodule), moving the main cystic components more centrally to facilitate biopsy.

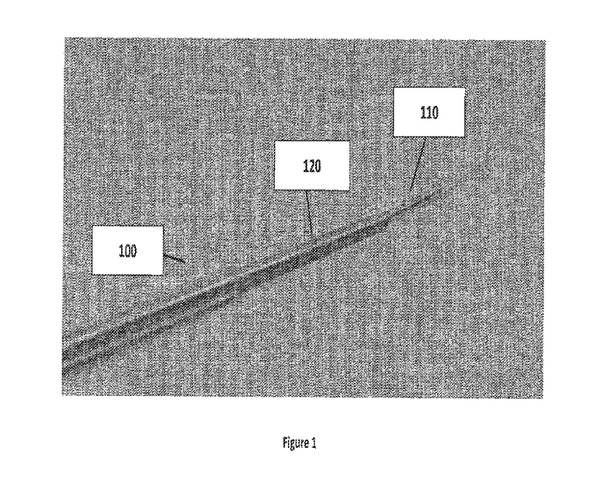

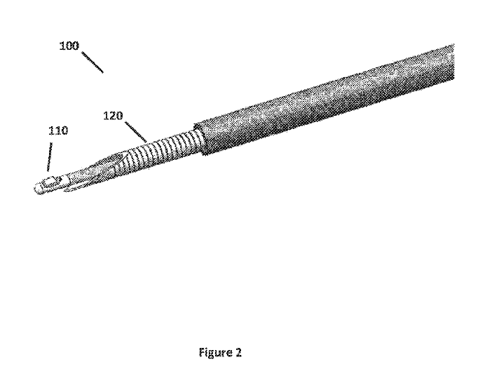

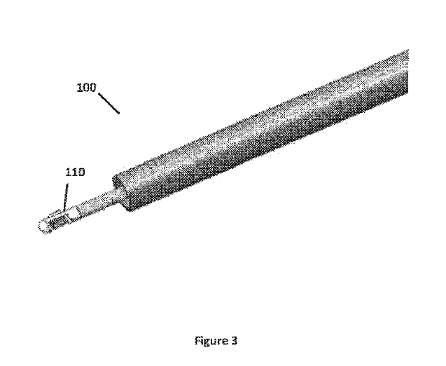

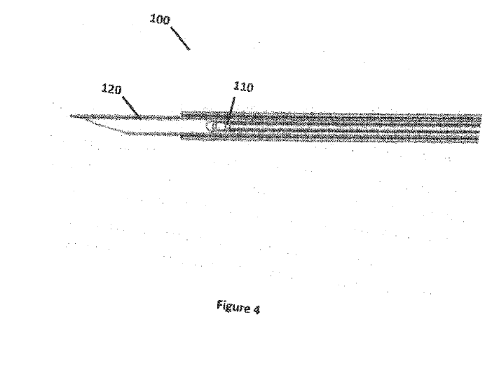

As discussed above, one of the most vexing difficulties in pulmonary nodule sampling is the deviation or deflection of catheter-based ultrasound probes and biopsy needles relative to one another due to differing stiffness of these instruments. This deflection is significantly reduced or even eliminated in systems according to the present disclosure, shown in use in prototype form in FIG. 1, and schematically in FIGS. 2-4. In an exemplary system 100 according to the present disclosure, an ultrasound probe 110 is slidably disposed within a biopsy needle 120 having a gauge of between 18 and 25, corresponding to an outer diameter of about 1.2 mm to about 0.5 mm, depending on the application. The needle 120 is, in turn, slidably disposed within a catheter or working channel of a scope, such as a bronchoscope, or a trocar, access sheath, cannula or other device used to access a tissue, organ or body cavity. In use, the needle 120 may be retracted relative to the probe 110 so the system 100 can be advanced, e.g., through the esophagus and bronchi as shown in FIG. 3. Once the system 100 is positioned near a tissue to be biopsied, such as a pulmonary nodule, the needle 120 is advanced and/or the ultrasound probe 110 is retracted to create a space within the lumen of the needle 120 into which a tissue sample can be taken. The needle 120 is then actuated, the sample is acquired and the needle 120 is withdrawn to expel the sample, and optionally reinserted to acquire another sample.

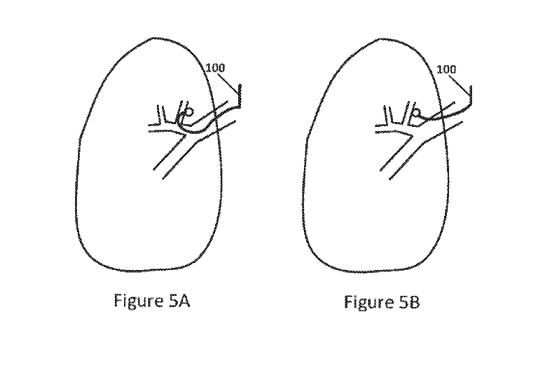

Turning now to FIGS. 5A and 56, the stiffness of the needle/probe arrangement described above makes it possible to take more direct “tunneling” approaches to pulmonary nodules or other tissues shown in FIG. 56. In contrast to the currently-used methods, schematized in FIG. 5A, which rely on navigation through potentially tortuous anatomy, such as bronchi, and which are characterized by the divergence between needle and probe described above, the tunneling approach is more direct, and allows the probe and needle to remain substantially coaxial throughout the procedure. The directness of such tunneling approaches, in turn, can reduce the time and difficulty of nodule sampling procedures. For clarity, though, the systems of the present disclosure can also be used to take more indirect, tortuous approaches illustrated in FIG. 5A, and their suitability for such approaches may be increased in some cases by increasing the flexibility of the needle 120.

It should be appreciated that, while the foregoing examples have focused on pulmonary nodule sampling, the systems and methods of the present disclosure are useful in any number of ultrasound-guided fine needle biopsy settings, including endoscopic ultrasound guided fine needle aspiration (EUS-FNA), as well as biopsies in the intestine, stomach, prostate, bladder, uterus, esophagus, etc.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present disclosure are described above. It is, however, expressly noted that the present disclosure is not limited to those embodiments, but rather the intention is that additions and modifications to what was expressly described herein are also included within the scope of the disclosure. Moreover, it is to be understood that the features of the various embodiments described herein were not mutually exclusive and can exist in various combinations and permutations, even if such combinations or permutations were not made express herein, without departing from the spirit and scope of the disclosure. In fact, variations, modifications, and other implementations of what was described herein will occur to those of ordinary skill in the art without departing from the spirit and the scope of the disclosure. As such, the disclosure is not to be defined only by the preceding illustrative description.

1. A system, comprising:
a biopsy needle having a gauge between 15 and 25; and
an ultrasound probe slidably disposed within a lumen of the biopsy needle.

2. The system according to claim 1, further comprising a device selected from the group consisting of an endoscope, a trocar, a cannula, and an access sheath, the device defining a lumen sized to permit insertion of the biopsy needle.

3. The system according to claim 1, wherein the ultrasound probe emits ultrasound having frequencies between 1 MHz and 400 MHz.

4. The system according to claim 3, wherein the probe emits ultrasound having frequencies between 20 and 400 MHz.

5. The system according to claim 1, wherein at least one of the biopsy needle and the ultrasound probe includes a radiopaque material.

6. The system according to claim 5, wherein the ultrasound probe comprises a radiopaque material.

7. The system according to claim 1, wherein at least one of the biopsy needle and the ultrasound probe is disposable.

8. A method of treating a patient, comprising the steps of:
inserting a biopsy needle and ultrasound probe through a bronchoscope into a bronchial tube of a patient, wherein the ultrasound probe is slidably disposed within a lumen of the biopsy needle;
advancing the ultrasound probe and the biopsy needle to a site where a biopsy is desired;
retracting the ultrasound probe into the biopsy needle, thereby creating a space into which a tissue sample can fit within the biopsy needle, and
actuating the biopsy needle, thereby acquiring a tissue sample in the space.

9. The method of claim 8, wherein the step of advancing the ultrasound probe and biopsy needle to a site where a biopsy is desired includes advancing the biopsy needle and ultrasound probe through a wall of the bronchial tube and through a lung tissue.

10. The method of claim 9, wherein the ultrasound probe is disposed inside the biopsy needle during the step of advancing the ultrasound probe and biopsy needle to the site where a biopsy is desired.

11. The method of claim 9, wherein the step of advancing the ultrasound probe and biopsy needle to a site where a biopsy is desired includes visualizing the biopsy needle under fluoroscopy.

12. The method of claim 9, wherein the step of advancing the ultrasound probe and biopsy needle to a site where biopsy is desired includes visualizing the site where biopsy is desired using an ultrasound signal generated by the ultrasound probe.

13. The method of claim 8, wherein the site where biopsy is desired is a pulmonary nodule.

14. The method of claim of claim 13, wherein the step of advancing the ultrasound probe and biopsy needle to a site where a biopsy is desired includes contacting one of the pulmonary nodule and a lung tissue near the pulmonary nodule with the biopsy needle, thereby altering a shape of the pulmonary nodule.

15. A system, comprising:
a bronchoscope having a working channel;
a biopsy needle slidably disposable within the working channel; and
an ultrasound probe slidably disposable in a lumen of the biopsy needle.

16. The system according to claim 15, wherein the ultrasound probe emits ultrasound having frequencies between 20 and 400 MHz.

17. The system according to claim 15, wherein at least one of the biopsy needle and the ultrasound probe is disposable.

18. The system according to claim 15, wherein at least one of the biopsy needle and the ultrasound probe includes a radiopaque material.

19. The system according to claim 15, wherein the biopsy needle has a stiffness greater than a stiffness of the ultrasound probe.

20. The system according to claim 15, wherein the biopsy needle has a gauge between 15 and 25.
